(19) United States
(12) Patent Application Publication
(10) Pub. No.: US 2015/0104520 A1
(43) Pub. Date: Apr. 16, 2015

(54) Nanoparticle Formulations in Biomarker Detection

(71) Applicants: Vuong Trieu, Agoura Hills, CA (US); Larn Hwang, Arcadia, CA (US)

(72) Inventors: Vuong Trieu, Agoura Hills, CA (US); Larn Hwang, Arcadia, CA (US)

(21) Appl. No.: 14/246,366

(22) Filed: Apr. 7, 2014

Related U.S. Application Data
(63) Continuation of application No. 61/853,440, filed on Apr. 5, 2013.

(51) Int. Cl. A61K 31/282; A61K 9/107; A61K 31/337

(52) U.S. Cl. CPC A61K 31/282; A61K 31/337; A61K 9/1075
USPC 424/490; 514/449

(57) ABSTRACT

The present invention relates to compositions and methods of treatment of cancer patients with cytotoxic drugs in particular the use of cytotoxic drugs encapsulated in a diblock copolymer formulation where the composition is stable in protein free media and less stable in protein containing media such as serum, in particular the treatment of ovarian.

### CROSS-REFERENCE TO RELATED APPLICATIONS

This application claims the benefit of priority to U.S. Provisional Application No. 61/853,440 filed Apr. 5, 2013 which is incorporated by reference in its entirety.

### STATEMENT REGARDING FEDERALLY SPONSORED RESEARCH OR DEVELOPMENT

Not applicable.

### FIELD OF THE INVENTION

The present invention relates to conditionally stable compositions and methods of treatment of cancer patients, including ovarian cancer patients with cytotoxic drugs in particular the use of cytotoxic drugs encapsulated in a diblock copolymer formulation where the composition is stable in protein free media and less stable in protein containing media such as serum.

### BACKGROUND OF THE INVENTION

Cancer is a leading cause of death worldwide, accounting for 7.6 million deaths (around 13% of all deaths) in 2008. Prostate cancer is a leading cause of cancer among men. Ovarian cancer is the ninth most common cancer in women and the fifth leading cause of cancer-related deaths in women in the US. One of every 72 women will develop ovarian cancer and one of every 100 will die from this form of cancer. The American Cancer Society estimates that in 2013 22,240 women will be diagnosed with ovarian cancer and about 14,230 will dies from ovarian cancer. About 85% to 90% of ovarian cancers are epithelial ovarian carcinomas. World-wide ovarian cancer is the seventh leading cause of death in women and uterine cancer is the fifth leading cause of death.

Treatment options include surgery, chemotherapy, and occasionally radiation therapy. Surgery usually involves the removal of one or both ovaries, fallopian tubes and the uterus. In advanced disease, surgically removing all abdominal metastases enhances the effect of chemotherapy and helps improve survival. For women with stage III ovarian cancer in which removal of cancerous tissue has been performed, studies show that chemotherapy administered both intravenously and directly into the peritoneal cavity improves survival.

Abraxane and Taxol are chemotherapeutic drugs. Both drugs are used to treat breast cancer. These cytotoxic medicines arrest the growth of cells in case of cancerous tissues. They essentially differ in the component they carry and their effectiveness. Taxol is an antineoplastic drug used in chemotherapy. It is an alkaloid derived from plants and prevents microtubule formation in cells. The drug has proven effects on breast, ovarian, bladder, prostate, esophageal, lung and melanoma cancers. The drug is solvent based and should be carefully administered since it is an irritant. The dosage and duration of administration of drug depends on the Body Mass Index and the severity of the disease. Side effects are common although the symptoms are either one or two in most cases. The most common side effects include hair loss, peripheral neuropathy, vomiting, diarrhea, myalagia, arthralagia, low blood counts and hypersensitivity.

Taxol is a first generation paclitaxel formulation in which Cremophor EL (polyoxyethylated castor oil) is mixed with paclitaxel and given as an infusion for the treatment of ovarian cancer, lung cancer, head and neck cancer, bladder cancer. Taxol has adverse side effects such as anaphylactic shock. Abraxane is paciltaxel nanoparticle encapsulated by albumin. The delivery of drug to target cells is easier when not formulated with solvent such as Cremophor. The drug is built on a natural albumin platform devoid of chemical solvents and there is little need for the concomitant or prior medications with anti-hypersensitive drugs. Abraxane is the drug of choice in first and second line of treatment in metastatic breast cancer and is approved in majority of the countries. Side effects of Abraxane include bone marrow suppression (primarily neutropenia) which is dose-dependent and a dose-limiting toxicity of Abraxane. In clinical studies, Grade 3-4 neutropenia occurred in 34% of patients with metastatic breast cancer (MBC) and 47% of patients with non-small cell lung cancer (NSCLC). Abraxane also requires tedious reconstitution which may cause foaming or clumping of the reconstituted lyophilized powder.

Biodegradable polymeric micelle-type drug compositions, containing a water-soluble amphiphilic block copolymer micelle having a hydrophilic poly(alkylene oxide) component and a hydrophobic biodegradable component, can be used to develop formulations in which a hydrophobic drug is physically trapped in the micelle. This micelle-type composition, enveloping a hydrophobic drug, can solubilize the hydrophobic drug in a hydrophilic environment to form a solution. IG-001 is a polymer bound nanoparticle paclitaxel and can be used to treat difficult to perfuse hypoxic tumors such as pancreatic cancer and ovarian cancer.

While there are cytotoxic drug compositions that are useful in the treatment of various cancers there exists a need for methods of treatment and drug administration that reduce side effects, have improved stability and/or increase the efficacy of the treatment regimen.

### SUMMARY OF THE INVENTION

The present invention relates to methods of treating patients with ovarian cancer by administering paclitaxel-containing micelles and carboplatin to patients. The invention relates to methods of treating patients with ovarian cancer by administering paclitaxel-containing micelles and carboplatin to patients where the micelles are comprised of a diblock copolymer including IG-001 which is a paclitaxel-containing micelle. The invention relates to methods of treating patients with ovarian cancer by administering paclitaxel-containing micelles and carboplatin where the amount of paclitaxel administered is from about 100 mg/m² to about 500 mg/m² and the carboplatin is from about 2 AUC to about 10 AUC. In one embodiment the paclitaxel is administered at 260 mg/m² and the carboplatin is 5 AUC. The invention relates to methods of treating patients with ovarian cancer by administering paclitaxel-containing micelles and carboplatin where the paclitaxel containing micelles and carboplatin are administered in multiple cycles. In one embodiment the administration is 6 cycles over 3 weeks. The invention relates to methods of treating patients with ovarian cancer by administering paclitaxel-containing micelles and carboplatin where the overall response rate is greater than 70% or greater than 80% or greater than 90% or greater than 95% or where the overall response rate is about 70% to about 95%.

The methods of the present invention provide for point-of-care treatment and analysis that are also patient-centric and invite better compliance and patient participation in personalizing their cancer treatment. Plasma or serum samples obtained via repetitive venipuncture represent the accepted gold standard for monitoring the pharmacodynamics (PD) and pharmacokinetics (pK) of anticancer agents. Unfortunately, the burden of frequent venipuncture is high; and, for researchers, venipuncture samples require immediate processing and storage facilities with freezers. The methods of the present invention resolve the operational difficulty associated with personalized therapy in general and paclitaxel treatment in specific.

The present invention relates to methods of treatment of cancer patients with cytotoxic drugs in particular the use of cytotoxic drugs encapsulated in a diblock copolymer formulation where the level of cytotoxic drug within the patient is monitored Based on the results of monitoring the drug the treatment regimen of the patient may be altered to increase the effectiveness of the drug treatment or decrease the toxicity of treatment or to monitor the effectiveness of the current drug regimen. The methods of the present invention permit self-monitoring of the cytotoxic drug and/or biomarkers.

The present invention relates to methods of improving paclitaxel therapy for cancer patients by treating patients with a first dosage of a paclitaxel formulation and monitoring the paclitaxel level in the patient such that the optimum dosage for additional dosages paclitaxel can be determined and utilized in the additional treatment regimens.

The methods of the present invention include monitoring of the biomarkers or paclitaxel levels by the patient or by the physician either in the patients home or in the physicians' office. The methods of monitoring and administration can be repeated to optimize treatment of the patient. The methods of the present invention provide for monitoring cancer drugs, such as paclitaxel, and biomarker levels from the patient's blood, urine or serum or any other appropriate means.

The present invention relates to methods of treatment of cancer patients with cancer treatment drugs including but not limited to cytotoxic drugs where the level of drug within the patient is monitored and the levels of biomarkers within the patient may also be monitored such that the treatment regimen of the patient may be altered to increase the effectiveness of the drug treatment. The methods of the present invention permit self-monitoring of the levels of cancer treatment drugs including cytotoxic drugs and/or biomarkers within a patient.

The present invention relates to methods of treatment of cancer patients with cytotoxic drugs in particular the use of cytotoxic drugs encapsulated in a diblock copolymer formulation where the level of cytotoxic drug within the patient is monitored and the levels of biomarkers within the patient are monitored. Based on the results of monitoring the drug and/or biomarkers the treatment regimen of the patient may be altered to increase the effectiveness of the drug treatment or decrease the toxicity of treatment or to monitor the effectiveness of the current drug regimen. The methods of the present invention permit self-monitoring of the cytotoxic drug and/or biomarkers.

The present invention relates to monitoring biomarkers such as, but not limited to luteinizing hormone (LH), B-type natriuretic peptide (BNP), Follicle Stimulating Hormone (FSH) as well as paclitaxel as prognostic indicators of response. The present invention relates to monitoring serum levels of biomarkers such as but not limited to luteinizing hormone (LH), B-type natriuretic peptide (BNP), Follicle Stimulating Hormone (FSH) as well as blood levels of paclitaxel as prognostic indicators of response.

The present invention relates to methods of improving paclitaxel therapy for cancer patients by treating patients with a first dosage of a paclitaxel formulation and monitoring the paclitaxel level in the patient such that the optimum dosage for additional dosages paclitaxel can be determined and utilized in the additional treatment regimens.

### DETAILED DESCRIPTION OF THE INVENTION

The present invention relates to methods of treating patients with ovarian cancer by administering paclitaxel-containing micelles and carboplatin to patients. The invention relates to methods of treating patients with ovarian cancer by administering paclitaxel-containing micelles and carboplatin to patients where the micelles are comprised of a diblock copolymer including IG-001 which is a paclitaxel-containing micelle. The invention relates to methods of treating patients with ovarian cancer by administering paclitaxel-containing micelles and carboplatin. In certain embodiment of the methods of the present invention the amount of paclitaxel administered may be greater than about 100 mg/m², or greater than about 150 mg/m², or greater than about 200 mg/m², or greater than about 225 mg/m², or greater than about 250 mg/m², or greater than about 300 mg/m², or greater than about 325 mg/m², greater than about 350 mg/m², or greater than about 375 mg/m², or greater than about 400 mg/m², or greater than about 450 mg/m². In certain embodiment of the methods of present invention the amount of paclitaxel administered may be from about 100 mg/m² to about 500 mg/m², or from about 100 mg/m² to about 450 mg/m², or from about 100 mg/m² to about 400 mg/m², or from about 100 mg/m² to about 350 mg/m², or from about 100 mg/m² to about 325 mg/m², or from 100 mg/m² to about 300 mg/m², or from about 100 mg/m² to about 275 mg/m², or from about 100 mg/m² to about 250 mg/m², or from about 100 mg/m² to about 200 mg/m², or from about 100 mg/m² to about 200 mg/m², or from about 100 mg/m² to about 150 mg/m², or from 150 mg/m² to about 500 mg/m² from about 150 mg/m² to about 450 mg/m², or from about 150 mg/m² to about 400 mg/m², or from about 150 mg/m² to about 375 mg/m², or from about 150 mg/m² to about 350 mg/m², or from about 150 mg/m² to about 325 mg/m², or from 150 mg/m² to about 300 mg/m² from about 150 mg/m² to about 275 mg/m², or from about 150 mg/m² to about 250 mg/m², or from about 150 mg/m² to about 225 mg/m², or from about 150 mg/m² to about 200 mg/m², or 200 mg/m² to about 500 mg/m², or from about 200 mg/m² to about 450 mg/m², or from about 200 mg/m² to about 400 mg/m², or from about 200 mg/m² to about 350 mg/m², or from about 200 mg/m² to about 325 mg/m², or from 200 mg/m² to about 300 mg/m², or from about 200 mg/m² to about 275 mg/m², or from about 200 mg/m² to about 250 mg/m², or from about 225 mg/m² to about 500 mg/m², or from about 225 mg/m² to about 450 mg/m², or from about 225 mg/m² to about 400 mg/m², or from 225 mg/m² to about 375 mg/m² from about 225 mg/m² to about 350 mg/m², or from about 225 mg/m² to about 300 mg/m², or from about 225 mg/m² to about 275 mg/m², or from about 225 mg/m² to about 250 mg/m², or from about 225 mg/m² to about 500 mg/m², or from 225 mg/m² to about 450 mg/m² from about 225 mg/m² to about 400 mg/m², or from about 225 mg/m² to about 375 mg/m², or from about 225 mg/m² to about 350 mg/m², or from about 225 mg/m² to about 325 mg/m², or 225 mg/m² to about 300 mg/m², or from about 225 mg/m² to about 275 g/m², or from about 225 mg/m² to about 250 mg/m², or from about 250 mg/m² to about 500 mg/m², or from about 250 mg/m² to about 450 mg/m², or from 250 mg/m² to about 400 mg/m², or from about 250 mg/m² to about 375 mg/m², or from about 250 mg/m² to about 350 mg/m², or from about 250 mg/m² to about 325 mg/m², or from about 250 mg/m² to about 300 mg/m², or from about 250 mg/m² to about 275 mg/m², or from 275 mg/m² to about 500 mg/m² from about 275 mg/m² to about 450 mg/m², or from about 275 mg/m² to about 400 mg/m², or from about 275 mg/m² to about 375 mg/m², or from about 275 mg/m² to about 350 mg/m², or from about 275 mg/m² to about 325 mg/m², or from 275 mg/m² to about 300 mg/m² from about 300 mg/m² to about 500 mg/m², or from about 300 mg/m² to about 450 mg/m², or from about 300 mg/m² to about 425 mg/m², or from about 300 mg/m² to about 400 mg/m², or from about 300 mg/m² to about 375 mg/m², or from about 300 mg/m² to about 350 mg/m², or from about 300 mg/m² to about 325 mg/m², or from about 325 mg/m² to about 500 mg/m², or from about 325 mg/m² to about 450 mg/m², or from 325 mg/m² to about 400 mg/m², or from about 325 mg/m² to about 375 mg/m², or from about 325 mg/m² to about 350 mg/m², or from about 350 mg/m² to about 500 mg/m², or from about 350 mg/m² to about 450 mg/m², or from about 350 mg/m² to about 425 mg/m², or from 350 mg/m² to about 400 mg/m² from about 350 mg/m² to about 375 mg/m², or from about 375 mg/m² to about 500 mg/m², or from about 375 mg/m² to about 450 mg/m², or from about 375 mg/m² to about 425 mg/m², or from about 375 mg/m² to about 400 mg/m², or from 400 mg/m² to about 500 mg/m² from about 400 mg/m² to about 450 mg/m², or from about 400 mg/m² to about 425 mg/m², or from about 150 mg/m² to about 225 mg/m², or from about 150 mg/m² to about 200 mg/m², or from about 220 mg/m² to about 300 mg/m², or from about 220 mg/m² to about 260 mg/m². The carboplatin may be delivered at at least 1 AUC, or at least 2 AUC, or at least 3 AUC, or at least 4 AUC or at least 5 AUC or at least 6 AUC or at least 7 AUC or at least 8 AUC or at least 9 AUC or at least 10 AUC. In other embodiments the carboplatin can be delivered at from about 1 AUC to about 10 AUC, or from about 1 AUC to about 9 AUC or from about 1 AUC to about 8 AUC, or from about 1 AUC to about 7 AUC, or from about 1 AUC to about 6 AUC, or from about 1 AUC to about 5 AUC, or from about 2 AUC to about 10 AUC of from about 2 AUC to about 9 AUC or from about 2 AUC to about 8 AUC, or from about 2 AUC to about 7 AUC, or from about 2 AUC to about 6 AUC, or from about 2 AUC to about 5 AUC, or from about 3 AUC to about 10 AUC of from about 3 AUC to about 9 AUC or from about 3 AUC to about 8 AUC or from about 3 AUC to about 7 AUC, or from about 3 AUC to about 6 AUC, or from about 3 AUC to about 5 AUC, or from about 4 AUC to about 10 AUC of from about 4 AUC to about 9 AUC or from about 4 AUC to about 8 AUC, or from about 4 AUC to about 7 AUC, or from about 4 AUC to about 6 AUC, or from about 4 AUC to about 5 AUC, or from about 5 AUC to about 10 AUC of from about 5 AUC to about 9 AUC or from about 5 AUC to about 8 AUC, or from about 5 AUC to about 7 AUC, or from about 5 AUC to about 6 AUC, or from about 6 AUC to about 10 AUC, or from about 6 AUC to about 9 AUC or from about 6 AUC to about 8 AUC. The invention relates to methods of treating patients with ovarian cancer by administering paclitaxel-containing micelles and carboplatin where the paclitaxel containing micelles and carboplatin are administered in multiple cycles over a period of time. The number of cycles may be more than 1, more than 2, more than 3, more than 4, more than 5, more than 6, more than 7, more than 8, more than 9 more than 10, more than 15, or more than 20. The number of cycles may be between from about 2 to about 10, or from about 2 to about 9 or from about 2 to about 8, or from about 2 to about 7, or from about 2 to about 6, or from about 2 to about 5, or from about 2 to about 4 or from about 2 to about 3, or from about 3 to about 10, or from about 3 to about 9 or from about 3 to about 8, or from about 3 to about 7, or from about 3 to about 6, or from about 3 to about 5, or from about 3 to about 4, or from about 4 to about 10, or from about 4 to about 9 or from about 4 to about 8, or from about 4 to about 7, or from about 4 to about 6, or from about 4 to about 5, or from about 5 to about 10 or from about 5 to about 9, or from about 5 to about 8, or from about 5 to about 7, or from about 5 to about 6, or from about 6 to about 10, or from about 6 to about 9 or from about 6 to about 8, or from about 6 to about 7. In one embodiment the combination of paclitaxel and carboplatin are administered in 6 cycles over 3 weeks. The combination of paclitaxel and carboplatin may be administered over a period of a day, or 2 days, or three days, or four days or five days or six days or a week, or two weeks or three weeks or a month or two months or longer. The invention relates to methods of treating patients with ovarian cancer by administering paclitaxel-containing micelles and carboplatin where the overall response rate is greater than 70% or greater than 80% or greater than 90% or greater than 95% or where the overall response rate is about 70% to about 95%.

Problems with current cancer treatments using cancer treatment drugs including cytotoxic drugs include high toxicity of the drug, lack of patient compliance, lack of feedback concerning the progress of the treatment regimen and lack of flexibility in designing a treatment regimen that is personalized for a given patient. The methods of the present invention provide for point-of-care treatment and analysis that are also patient-centric and invite better compliance and patient participation in personalizing their cancer treatment.

Personalized dosing by point-of-care Therapeutic Drug Monitoring (TDM) includes a point-of-care device which allows for self-sampling and self-monitoring of the cancer treatment drug including paclitaxel level in a patient, in particular the blood level of paclitaxel. Personalized dosing reduces toxicity while increasing efficacy of the treatment. The rapid and quantitative point-of-care lateral flow device permits testing of bodily fluids to provide information on paclitaxel levels to allow for personalized dosing (Pac-TDM). The present invention relates to methods of improving paclitaxel therapy for cancer patients by treating patients with a first dosage of a cancer treatment drug formulation and monitoring the drug level in the patient such that the optimum dosage for additional dosages paclitaxel may be determined and the patient treated accordingly. For example, a given patient may be diagnosed with a particular cancer and a regimen of cytotoxic drug therapy such as paclitaxel is prescribed. Based upon the timing of the treatment the patient or the physician can test the patient to determine the level of paclitaxel in the patient. If the concentration of paclitaxel is determined to be too high or too low the treatment regimen, including the timing of administration of the drug and the dosage of the drug can be altered to optimize the treatment. Determining the level of cancer treatment drug may need to be repeated one or more times to make sure the optimal treatment regimen is applied. Components to the personalized paclitaxel therapy may include, but is not limited to the paclitaxel formulation, personalized dosing by TDM, biomarker testing and pK testing.

Problems with current cancer treatments using cancer treatment drugs including cytotoxic drugs include high toxicity of the drug, lack of patient compliance, lack of feedback concerning the progress of the treatment regimen and lack of flexibility in designing a treatment regimen that is personalized for a given patient. The methods of the present invention provide for point-of-care treatment and analysis that are also patient-centric and invite better compliance and patient participation in personalizing their cancer treatment. Plasma or serum samples obtained via repetitive venipuncture represent the accepted gold standard for monitoring the pharmacodynamics (PD) and pharmacokinetics (PK) of anticancer agents. Unfortunately, the burden of frequent venipuncture is high; and, for researchers, venipuncture samples require immediate processing and storage facilities with freezers. The methods of the present invention resolve the operational difficulty associated with personalized therapy in general and paclitaxel treatment in specific. The methods of the present invention solve these issues by providing rapid and quantitative point-of-care tests for pharmacodynamics (PD) biomarker testing and paclitaxel pharmacokinetic (pK) therapeutic drug monitoring. The methods of the present invention may eliminate the burden of frequent venipuncture; eliminate the need for well-equipped processing and storage facilities; are useful at home, primary care physician's office, or remote locations where sophisticated equipment is not available. Moreover, no dilution or manipulation of the patient sample is required due to the expanded dynamic range of the technology utilized in the present methods. The devices utilized for testing permit self-sampling and self-testing.

Patient selection by point-of-care biomarker testing utilizes a point-of-care device to identify patients who are responders or non-responders to the current treatment by determining the level of certain biomarkers in response to the treatment. The rapid and quantitative point-of-care lateral flow device permits testing of bodily fluids to provide information on biomarker levels to allow for biomarker testing (PD biomarkers, predictive and prognostic biomarker testing). For example, a given patient may be diagnosed with a particular cancer and a regimen of cytotoxic drug therapy such as paclitaxel is prescribed. Based upon the timing of the treatment the patient or the physician can test the patient to determine the level of particular biomarkers in the patient. The level of biomarker within the patient may be indicative as to whether the treatment is effective. In other words the biomarker level can determine which patient is responding to the treatment and which patient is not. The treatment regimen including the timing of administration of the drug and the dosage of the paclitaxel can be altered to optimize the treatment. In some cases, it may be advisable to change the type of cytotoxic drug given the patient. Determining the level of different biomarkers may need to be repeated one or more times to make sure the optimal treatment regimen is applied.

The lateral flow assay may be constructed by using mAb pairs against a given marker such as FSH, LH, BNP, and paclitaxel. Quantitation may be determined by a POC reflectometric optical reader, which utilizes confocal optics with a low distance-to-target ratio. Calculations were performed in the background using information embedded on the 2D-bar code specific to each lot of cassettes. Serum/plasma, urine, blood or any other suitable fluid may be used to determine marker levels.

Biomarkers may include but are not limited to FSH, LH, and BNP. Combination of one or more biomarkers may be used.

The micellular formulations of the present invention are quite stable in protein-free media. Sustained release micelles have been prepared in which polymers with very low CMC (<0.1 μg/ml) can be used for prolonging the circulation time before the micelle degrades. Upon intravenous injection, the micelles undergo dilution in the body. If the CMC of the micelles is high, the concentration of the polymer or surfactant falls below the CMC upon dilution and hence, the micelles dissociate. Therefore, a higher concentration of the polymer or surfactant has to be used to prepare the micelles so that they withstand the dilution up to 5 l in the blood. However, the use of high concentrations might not be feasible due to toxicity related dose limitations. If the polymer or surfactant has a CMC lower than 0.1 μg/ml, concentrations as low as 5 mg/ml may be used to prepare a micelle formulation in order to counter the dilution effects in the blood. A variety of polymers including diblock copolymers, triblock copolymers and graft copolymers have been synthesized to be stable even after intravenous administration. The formulations of the present invention provide methodologies for constructing formulations in which the nanoparticles are less stable once administered such that the drug compound can be released from the nanoparticle and made available to the endogenous albumin delivery system. Nanoparticles of the present invention are more stable in protein-free solutions than in solutions containing proteins such as serum. The nanoparticles of the present invention may be at least 20% more stable or 25% more stable or 30% more stable or 35% more stable or 40% more stable or 45% more stable or 50% more stable or 55% more stable or 60% more stable or 65% more stable or 70% more stable or 75% more stable or 80% more stable or 85% more stable or 90% more stable or 95% more stable or 100% more stable or 125% more stable or 150% more stable or 175% more stable or 200% more stable or 500% more stable or 1000% more stable or 5000% more stable or 10000% more stable in a protein free solution than in a solution containing protein. The nanoparticles of the present invention may be between about 10% more stable to about 25000% more stable or about 10% more stable to about 15000% more stable or about 10% more stable to about 12500% more stable or about 10% more stable to about 10000% more stable or from about 10% more stable to about 9000% more stable or from about 10% more stable to about 8000% more stable or from about 10% more stable to about 7000% more stable or from about 10% more stable to about 6000% more stable or from about 1000% more stable to about 500% more stable or from about 10% more stable to about 400% more stable or from about 10% more stable to about 300% more stable or about 10% more stable to about 200% more stable or about 20% more stable to about 125% more stable or about 20% more stable to about 100% more stable or from about 20% more stable to about 90% more stable or from about 20% more stable to about 80% more stable or from about 20% more stable to about 70% more stable or from about 20% more stable to about 60% more stable or from about 20% more stable to about 50% more stable or from about 20% more stable to about 40% more stable or from about 50% more stable to about 2500% or from about 50% more stable to about 1250% more stable or from about 50% more stable to about 1000% more stable in a protein free solution than in a solution containing protein.

Nanoparticles of the present invention are less stable in solutions or media containing proteins such as serum than Abraxane. The nanoparticles of the present invention may be at least 20% less stable or 25% less stable or 30% less stable or 35% less stable or 40% less stable or 45% less stable or 50% less stable or 55% less stable or 60% less stable or 65% less stable or 70% less stable or 75% less stable or 80% less stable or 85% less stable or 90% less stable or 95% less stable or 100% less stable or 125% less stable or 150% less stable or 175% less stable or 200% less stable or 500% less stable or 1000% less stable or 5000% less stable or 10000% less stable in a protein free solution than in a solution containing protein. The nanoparticles of the present invention may be between about 10% less stable to about 25000% less stable or about 10% less stable to about 15000% less stable or about 10% less stable to about 12500% less stable or about 10% less stable to about 10000% less stable or from about 10% less stable to about 9000% less stable or from about 10% less stable to about 8000% less stable or from about 10% less stable to about 7000% less stable or from about 10% less stable to about 6000% less stable or from about 1000% less stable to about 500% less stable or from about 10% less stable to about 400% less stable or from about 10% less stable to about 300% more stable or about 10% more stable to about 200% more stable or about 20% more stable to about 125% more stable or about 20% more stable to about 100% more stable or from about 20% more stable to about 90% more stable or from about 20% more stable to about 80% more stable or from about 20% more stable to about 70% more stable or from about 20% more stable to about 60% more stable or from about 20% more stable to about 50% more stable or from about 20% more stable to about 40% less stable or from about 50% less stable to about 2500% or from about 50% less stable to about 1250% less stable or from about 50% less stable to about 1000% less stable in a protein free solution than in a solution containing protein.

The methods of the present invention may be used with any formulation of cancer treatment drugs including but not limited to polymeric micelle nanoparticle formulations include amphiphilic block copolymer which may comprise a hydrophilic block (A) and a hydrophobic block (B) linked with each other in the form of A-B, A-B-A or B-A-B structure. Additionally, the amphiphilic block copolymer may form core-shell type polymeric micelles in its aqueous solution state, wherein the hydrophobic block forms the core and the hydrophilic block forms the shell.

In one embodiment, the hydrophilic block (A) of the amphiphilic block copolymer may be polyethylene glycol (PEG) or monomethoxypolyethylene glycol (mPEG). Particularly, it may be mPEG. The hydrophilic block (A) may have a weight average molecular weight of 500-20,000 daltons, specifically 1,000-5,000 daltons, and more specifically 1,000-2,500 daltons.

The hydrophobic block (B) of the amphiphilic block copolymer may be a water-insoluble, biodegradable polymer. In one embodiment, the hydrophobic block (B) may be polylactic acid (PLA) or poly(lactic-co-glycolic acid) (PLGA). In another embodiment, the hydrophobic block (B) may have a weight average molecular weight of 500-20,000 daltons, specifically 1,000-5,000 daltons, and more specifically 1,000-2,500 daltons. Hydroxyl end groups of the hydrophobic block (B) may be protected with fatty acid groups, and particular examples of the fatty acid groups include acetate, propionate, butyrate, stearate, palmitate groups, and the like. The amphiphilic block copolymer comprising the hydrophilic block (A) and the hydrophobic block (B) may be present in the composition in an amount of 20-98 wt %, specifically 65-98 wt %, and more specifically 80-98 wt % based on the total dry weight of the composition.

In another embodiment, the hydrophilic block (A) and the hydrophobic block (B) may be present in the amphiphilic block copolymer in such a ratio that the copolymer comprises 40-70 wt %, specifically 50-60 wt % of the hydrophilic block (A) based on the weight of the copolymer. When the hydrophilic block (A) is present in a proportion less than 40%, the polymer has undesirably low solubility to water, resulting in difficulty in forming micelles. On the other hand, when the hydrophilic block (A) is present in a proportion greater than 70%, the polymer becomes too hydrophilic to form stable polymeric micelles, and thus the composition may not be used as a composition for solubilizing taxane.

A preferred paclitaxel formulation is IG-001 (also referred to as Genexol-PM, Cynviloq™) which is a cremophor-free, polymeric micelle formulation of paclitaxel. IG-001 (Genexol-PM) utilizes biodegradable di-block copolymer composed of methoxy poly (ethylene glycol)-poly (lactide) to form nanoparticles with paclitaxel containing hydrophobic core and a hydrophilic shell. The micellar composition may be made by dissolving an amphipathic co-polymer, monomethoxypolyethylene glycol-polylactide with an average molecular weight of 1766-2000 daltons at 80° C. in ethanol. Paclitaxel is added to the dissolved co-polymer and the solution cooled to about 50° C. where room temperature water is added. Anhydrous lactose may be added and dissolved. The solution may then be filtered and lyophilized. The amount of paclitaxel in the micelle formulation can be altered. Less or more paclitaxel will change the loading % and change the CMC and properties of the formulation. The size of the nanoparticles for IG-001 is a Gaussian distribution where the mean particle size is about 10 nm to about 50 nm.

Cytotoxic drug levels may include but are not limited to levels for paclitaxel, docetaxel, 7-epipaclitaxel, t-acetyl paclitaxel, 10-desacetyl-paclitaxel, 10-desacetyl-7-epipaclitaxel, 7-xylosylpaclitaxel, 10-desacetyl-7-glutarylpaclitaxel, 7-N,N-dimethylglycylpaclitaxel, 7-L-alanylpaclitaxel.

Cancer types for which the methods of the present invention may be useful include but are not limited to ovarian cancer, breast cancer, pancreatic cancer, liver cancer, non-small cell lung cancer (NSCLC) and other lung cancers.

### EXAMPLES

### Example 1

### Phase I Study

A multi-institutional phase I study was conducted in which the subject selection criteria included patients 18 years of age and older with epithelial ovarian cancer, FIGO stage IIIB-IV, primary whose life expectancy was 6 months or more and who could be treated with paclitaxel and carboplatin. The study utilized a novel Cremophor-free, polymeric micelle formulation of paclitaxel with carboplatin as a primary treatment in patients with advanced ovarian carcinoma was conducted to determine the Maximum Tolerated Dose (MTD) and dosing for Phase 2 trial of IG-001 in combination with carboplatin. Other objectives included overall survival, progress free survival, time to progression, duration of overall response and safety and toxicity. Six patients/dose level were treated with 220, 260, and 300 mg/in a manner shown in Table 1. MTD was not determined in this phase 1 trial (over 300 mg/m²).

TABLE 1

| | Total | Investigator | Best Overall Response |
|---|---|---|---|
| Group | Cycles | Evaluation | (Reviewer Evaluation) |
| 220 mg/m² | 6 cycle | PR | PR |
| | 6 cycle | CR | PR |
| | 6 cycle | PR | PR |
| | 2 cycle | PR | PR |
| | 6 cycle | CR | PR |
| | 6 cycle | PR | PR |
| 260 mg/m² | 6 cycle | PR | PR |
| | 6 cycle | PR | PR |
| | 6 cycle | PR | PR |
| | 2 cycle | PD | PD |
| | 6 cycle | CR | PR |
| | 6 cycle | CR | CR |
| 300 mg/m² | 1 cycle | NA | PR |
| | 6 cycle | PR | PR |
| | 2 cycle | PR | PR |
| | 6 cycle | CR | CR |
| | 6 cycle | CR | PR |
| | 6 cycle | PR | PR |

Dose Limiting Toxicity was shown as grade 4 myalgia in one patient at 300 mg/m². The result indicated a response rate of 94.12%, adverse events: 20.64% and LT toxicity: 7.83%. No death related to the disease or treatment. The recommended dose for Phase 2 trial of cremophor-free paclitaxel in combination with Carboplatin is 260 mg/m². Cremophor-free paclitaxel could be considered to be superior to conventional paclitaxel because of the possibilities of delivering higher paclitaxel dose without additional toxicities.

### Example 2

### Phase 2 Trial to Evaluate Efficacy and Safety of Combination Therapy of IG-001 Plus Carboplatin Compared to Paclitaxel Plus Carboplatin as a 1st Line Treatment in Patients with Epithelial Ovarian Cancer

Phase II ovarian trial design: randomized, two-arm trial, primary advanced epithelial ovarian cancer consisting of 100 patients (50/each arm). Control Arm: Solvent based paclitaxel 175 mg/m2 IV+Carboplatin 5 AUC IV, 3 weeks, 6 cycles and Experimental Arm: IG-001 260 mg/m² IV+Carboplatin 5 AUC IV 3 weeks, 6 cycles. The primary objective of the study was to determine the Composite Response by GCIG CA-125 Response and Response Evaluation Criteria in Solid Tumors (RECIST): based on non-inferiority study. Patients with FIGO stage IC-IV epithelial ovarian cancer after debulking surgery were split into two groups: a treatment group (treatment group: Genexol-PM 260 mg/m²+Carboplatin 5 AUC) and a control group (Paclitaxel 175 mg/m²+Carboplatin 5 AUC). The treatment regimen included 6 cycles of treatment with injection every three weeks and tumor screening after cycles 2, 4 and 6.

TABLE 2

| | Response n (%) | C.I. | Patients n(%) | p-value |
|---|---|---|---|---|
| Experiment (IG-001 + | 44 (88.00 | (80.44, 95.56) | 50 (51.02) | 0.7005 |
| Carboplatin | | | | |
| Control (Paclitaxel + Carboplatin) | 37 (77.08) | (67.10, 87.06) | 48 (48.98) | |
| Total | 81 (82.65 | (76.36, 88.95) | 98 (100.00) | |
| Experimental group - control | −10.92 | (−∞, 1.60) | | |
| group | | | | |

TABLE 3

| | | Rate (%) | Number (n) | Pt. No. n(%) | p-value |
|---|---|---|---|---|---|
| AE | IG-001 + | 50 (100.00) | 418 | 50 (51.02) | 0.0539 |
| | Carboplatin | | | | |
| | Paclitaxel + | 44 (91.67) | 360 | 48 (48.98) | |
| | Carboplatin | | | | |
| | Total | 94 (95.92) | 778 | 98 (100.00) | |
| SAE | IG-001 + | 20 (40.00) | 51 | 50 (51.02) | 0.3662 |
| | Carboplatin | | | | |
| | Paclitaxel + | 15 (31.25) | 37 | 48 (48.98) | |
| | Carboplatin | | | | |
| | Total | 35 (35.71) | 88 | 98 (100.00) | |
| UAE | IG-001 + | 1 (2.00) | 1 | 50 (51.02) | 1.0000 |
| | Carboplatin | | | | |
| | Paclitaxel + | 0 (0.00) | 0 | 48 (48.98) | |
| | Carboplatin | | | | |
| | Total | 1 (1.02) | 1 | 98 100.00) | |

The results indicate that the response rate was 88.00% vs. 77.8% (IG-001 vs. paclitaxel) and the primary endpoint of noninferiority to paclitaxel was met. The one-sided 95% upper confidence limit was 4.95, which is lower than the non-inferiority threshold (16.3%), indicating that the study group is not inferior to the control group. Adverse events were similar to paclitaxel despite the higher dose.

### Example 3

### Biomarker Concentrations in Healthy and Cancer Patients

A lateral flow assay was constructed using monoclonal antibody (mAb) pairs against Follicle Stimulating Hormone. Similar methods were used to develop the Lutenizing Hormone (LH), basic natriuretic peptide (BNP), and Paclitaxel point of care (POC) tests. Quantitation of the biomarkers was performed with a reflectometric optical reader, which utilizes confocal optics with a low distance-to-target ratio. Calculations were performed in the background using information embedded on the 2D-bar code specific to each lot of cassettes. Serum, urine from pre-puberty females, and blood from female donors were used as matrices for these experiments.

Serum samples collected at time of diagnosis of ovarian cancer were tested using rapid and quantitative point-of-care (POC) devices for blood biomarkers (CA125, FSH, BNP, and paclitaxel) and the data were evaluated using JMP9 statistical analysis software. 41 ovarian cancer patients were analyzed for cancer antigen 125 (CA125) prior to surgery of which 3 (7%), 11 (27%), and 27 (66%) were clear cell carcinoma, cystadenocarcinoma and adenocarcinoma, respectively. CA125 level was higher for clear cell histological subtype than other subtypes [clear cell (N=13; 1471±1307 U/mL), endometrioid (N=23; 574±442 U/mL), mucinous (N=16; 255±272 U/mL), and serous (N=83; 808±1337 U/mL)], p=0.005, p=0.0259, p=0.055 vs. mucinous, endometrioid, and serous subtypes, respectively. Because the serous subtype is the predominant subtype with a great deal of variability in CA-125, it was chosen for further examination for FSH, and BNP. FSH and BNP were quantitated using point-of-care devices. FSH was significantly higher for cancer patients than normal. The incidence of FSH positivity (>20 IU/L) was 17 of 19 (89%) among cancer patients vs. 4 of 10 (40%) among normal, p=0.0046. In the serous adenocarcinoma group, FSH level was higher (median=151.6 mU/ml) vs. normal controls (median of 13.4 mU/ml, p=0.01, Wilcoxon). FSH progressively increased from normal controls, to normotensive patients, to hypertensive patients with median FSH values of 13.4, 79.3, and 232.2, respectively. Incidence of BNP>25 pg/ml was higher for patients (14 of 19, 74%) vs. normal controls (3 of 10, 30%, p=0.02, Chi-square). FSH and BNP exhibited increased incidence/level among cancer patients versus age-matched normals. There were no correlations between CA125 and FSH or BNP suggesting that they are independent biomarkers.

TABLE 4

| BNP (pg/ml) Quantiles | 10% | 25% | Median | 75% | 90% |
|---|---|---|---|---|---|
| Normal | 0 | 0 | 0 | 84.1 | 177.3 |
| Ovarian Cancer | 0 | 0 | 40.0 | 74.8 | 127.3 |
| (p = .02 Chi Square) | | | | | |

TABLE 5

| FSH (IU/L) Quantiles | 10% | 25% | Median | 75% | 90% |
|---|---|---|---|---|---|
| Normal | 3.8 | 7.8 | 13.4 | 99.7 | 828.8 |
| Ovarian Cancer | 15.1 | 73.4 | 151.8 | 418.6 | 825.0 |
| (p = .01 Willcoxon) | | | | | |

Within this disclosure, any indication that a feature is optional is intended provide adequate support (e.g., under 35 U.S.C. 112 or Art. 83 and 84 of EPC) for claims that include closed or exclusive or negative language with reference to the optional feature. Exclusive language specifically excludes the particular recited feature from including any additional subject matter. For example, if it is indicated that A can be drug X, such language is intended to provide support for a claim that explicitly specifies that A consists of X alone, or that A does not include any other drugs besides X. “Negative” language explicitly excludes the optional feature itself from the scope of the claims. For example, if it is indicated that element A can include X, such language is intended to provide support for a claim that explicitly specifies that A does not include X. Non-limiting examples of exclusive or negative terms include “only,” “solely,” “consisting of,” “consisting essentially of,” “alone,” “without”, “in the absence of (e.g., other items of the same type, structure and/or function)” “excluding,” “not including”, “not”, “cannot,” or any combination and/or variation of such language.

Similarly, referents such as “a,” “an,” “said,” or “the,” are intended to support both single and/or plural occurrences unless the context indicates otherwise. For example “a dog” is intended to include support for one dog, no more than one dog, at least one dog, a plurality of dogs, etc. Non-limiting examples of qualifying terms that indicate singularity include “a single”, “one,” “alone”, “only one,” “not more than one”, etc. Non-limiting examples of qualifying terms that indicate (potential or actual) plurality include “at least one,” “one or more,” “more than one,” “two or more,” “a multiplicity,” “a plurality,” “any combination of,” “any permutation of,” “any one or more of,” etc. Claims or descriptions that include “or” between one or more members of a group are considered satisfied if one, more than one, or all of the group members are present in, employed in, or otherwise relevant to a given product or process unless indicated to the contrary or otherwise evident from the context.

Where ranges are given herein, the endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While this invention has been particularly shown and described with references to example embodiments thereof, it will be understood by those skilled in the art that the various changes in form and details may be made therein without departing from the scope of the invention encompassed by the appended claims.

1. A method of treating patients with ovarian cancer comprising administering paclitaxel containing micelles and carboplatin to the patient.

2. The method of claim 1 wherein the micelle is comprises of a diblock copolymer.

3. The method of claim 2 wherein the paclitaxel containing micelles are IG-001.

4. The method of claim 3 wherein the amount of paclitaxel administered is from about 220 mg/m² to 300 mg/m².

5. The method of claim 4 wherein the amount of palitaxel administered is about 260 mg/m².

6. The method of claim 5 wherein the carboplatin is about 4-8 AUC.

7. The method of claim 6 wherein the carboplatin AUC is about 5.

8. The method of claim 5 wherein the paclitaxel containing micelles and carboplatin are administered in at least 6 cycles.

9. The method of claim 8 wherein the paclitxel containing micelles and carboplatin are administered in 6 cycles over 3 weeks.

10. The method of claim 9 wherein the overall response rate is greater than 70%.

11. The method of claim 9 wherein the overall response rate is greater than 80%.

12. The method of claim 9 wherein the overall response rate is greater than 90%.

13. The method of claim 9 wherein the overall response rate is greater than 95%.

14. The method of claim 9 wherein the overall response rate is about 70% to about 95%.

15. A conditionally stable micelle composition containing an active compound wherein the composition is stable in protein-free medium and unstable in a protein containing medium.

16. The conditionally stable micelle composition of claim 15, wherein the composition has a higher maximum tolerated dose.

17. The conditionally stable micelle composition of claim 15, wherein the composition has improved intraperitoneal delivery.

18. The conditionally stable micelle composition of claim 15, wherein the composition has expanded dose proportionality.

19. The conditionally stable micelle of claim 15 wherein the micelle is comprises of a diblock copolymer.

20. The conditionally stable micelle of claim 15 wherein the active compound is paclitaxel.

21-50. (canceled)
